(19) United States
(12) Patent Application Publication
(10) Pub. No.: US 2025/0121187 A1
(43) Pub. Date: Apr. 17, 2025

(54) MONOTONIC AMPLITUDE PULSE TRAINS FOR NON-INVASIVE NEUROSTIMULATION

(71) Applicant: CARNEGIE MELLON UNIVERSITY, Pittsburgh, PA (US)

(72) Inventors: Chaitanya Goswami, Pittsburgh, PA (US); Mats Forssell, Pittsburgh, PA (US); Vishal Jain, Pittsburgh, PA (US); Maysam Chamanzar, Pittsburgh, PA (US); Pulkit Grover, Pittsburgh, PA (US)

(73) Assignee: CARNEGIE MELLON UNIVERSITY, Pittsburgh, PA (US)

(21) Appl. No.: 18/914,612

(22) Filed: Oct. 14, 2024

Related U.S. Application Data
(60) Provisional application No. 63/544,037, filed on Oct. 13, 2023.

(51) Int. Cl. A61N 1/36; A61N 1/04

(52) U.S. Cl. CPC A61N 1/36034; A61N 1/36025; A61N 1/0456; A61N 1/0476

(57) ABSTRACT

A method of delivering a non-invasive neurostimulation using a Monotonic Amplitude Pulse Train is more charge-efficient than using conventional, uniform waveforms. Monotonic Amplitude Pulse Trains optimize the amplitudes of individual pulses in a train to reduce the total energy required to provide neural stimulation by as much as 50%.

### RELATED APPLICATIONS

This application claims the benefit of U.S. Provisional Patent Application No. 63/544,037, filed Oct. 13, 2023, the contents of which are incorporated herein in their entirety.

### GOVERNMENT INTEREST

This invention was made with support of the United States government under contract N65236-19-C-8017, awarded by the Department of the Navy. The U.S. government has certain rights in the invention.

### FIELD OF THE INVENTION

This invention is related to the field of neural stimulation and, in particular, to non-invasive neural stimulation techniques, such as transcranial electrical stimulation (TES).

### BACKGROUND

Electrical current is one of the most widely used and safest modalities for stimulating neurons, either in the brain or the peripheral nerves. Electrical stimulation of neurons has shown promise in the treatment of various neurological diseases, such as reducing Parkinsonian symptoms, stroke rehabilitation, treating clinical depression, and many more.

Electrical currents can be delivered either invasively (i.e. by implanting an electrode in the brain or inside the body through a surgical operation), or non-invasively by placing electrodes on the scalp (in case of stimulating the brain) or on the skin (in the case of peripheral nerves).

A major advantage of non-invasive electrical stimulation is that it does not require a surgical procedure to implant an electrode, thereby mitigating the short-term complications that may be induced by surgery (e.g., infection), or longer-term complications that may be induced by the implanted electrode.

However, non-invasive electrical stimulation tends to be less precise than invasive stimulation and typically requires a larger current to induce neural activity. This is due to the fact that the current has to travel from the scalp/skin, typically through a bone (such as the skull in the case of stimulating the brain), to reach the neural target (typically the brain, spinal cord or a peripheral nerve). This results in a loss of the energy/current traveling from the electrodes to the neural target.

Traditionally, neural stimulation uses equiamplitude pulse trains of identical rectangular pulses, where pulse widths, amplitudes, and frequencies are tuned to meet experimental needs. To avoid damage to the scalp/skin, the energy/charge of the injected currents is restricted at the electrodes (on the scalp/skin). This severely impacts the ability of non-invasive stimulation to induce neural activity. For example, it is believed that transcranial direct current stimulation (TDCS) is not able to elicit direct neural activity, but rather works by modulating the synaptic strength of neurons.

Prior art efforts tend to focus on the case of invasive electrical stimulation by designing minimum energy waveforms for single neurons. Little attention has been paid in the art to designing minimal energy/charge waveforms for non-invasive stimulation. Therefore, it would be desirable to design waveforms that can be effective in the stimulation of various neural volumes in the brain when administered non-invasively.

### SUMMARY

Disclosed herein is a novel non-uniform pulse train shape, which is referred to herein as a Monotonic Amplitude Pulse Train (MAPT), that is more charge-efficient than conventional waveforms. Monotonic Amplitude Pulse Trains require as much as 50% less charge than conventional pulse trains to induce motor activity in non-invasive neurostimulation.

Optimizing the amplitudes of individual pulses in a train reduces the total energy required to provide neural stimulation, alleviating various conditions that can result from such stimulation (e.g., scalp pain from transcranial stimulation, risk of tissue damage, etc.).

### BRIEF DESCRIPTION OF THE DRAWINGS

By way of example, a specific exemplary embodiment of the disclosed system and method will now be described, with reference to the accompanying drawings, in which:

FIG. 1 is a graph of an example Monotonic Amplitude Pulse Train in accordance with the present invention.

FIG. 2 is a comparison of uniform amplitude, increasing monotonic and decreasing monotonic waveforms.

FIGS. 3(A-C) show different electrode montages for non-invasive delivery of a MAPT waveform with various levels of focality. The bottom plots show simulations of the electric field generated in the brain

FIGS. 4(A-B) are graphs showing the total charge injected and the maximum current injected, respectively, required to stimulate motor cortex (motor threshold) as a function of focus level.

FIGS. 5(A-C) are graphs showing the calculation of motor threshold determined from measurements of muscle activity for uniform, monotonically increasing and monotonically decreasing waveforms, respectively.

### DETAILED DESCRIPTION

One way to improve the efficacy of non-invasive stimulation is to design the temporal shape of the electrical waveform such that the amount of energy/charge required to stimulate the neural target is reduced. Disclosed herein is the design of energy/charge-efficient waveforms for non-invasive stimulation.

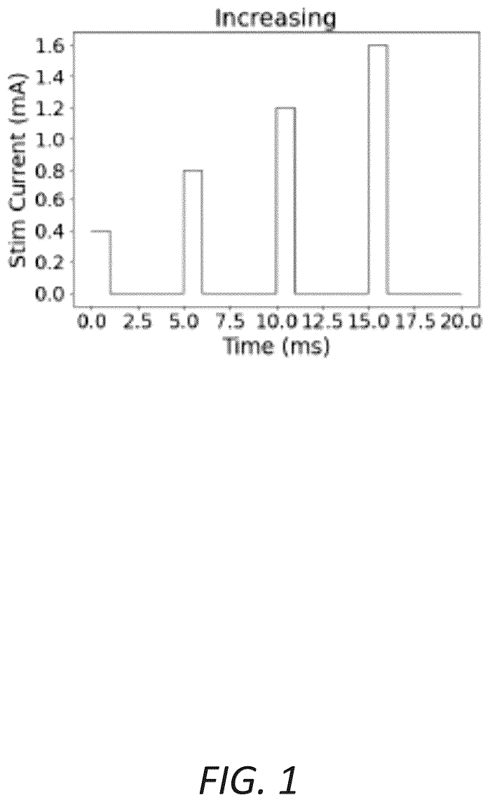

The novel Monotonic Amplitude Pulse Train of the present invention can be described as follows, and an example is presented in FIG. 1. First, the waveform is in the form of monophasic or biphasic pulses. In variations, the polarity of a monophasic waveform may be anodic or cathodic. In the case of a biphasic waveform, the waveform may be asymmetric (i.e., different amplitudes in each phase).

Each pulse is on for a fixed duration of W seconds, also known as the pulse width of the MAPT. In preferred embodiments, the range of W lies approximately between 10 μs and 10 ms. In other embodiments, other pulse widths can be used and are contemplated to be within the scope of the invention. In yet other embodiments, the width of each pulse may be different.

Each pulse is repeated at a frequency of f Hz. In preferred embodiments, the range of f lies between 1 Hz and 1 kHz, although in other embodiments other ranges may be used. In other embodiments, the frequency may vary between pulses, that is, the pulses may not be evenly spaced in time.

Although the pulses can be of any shape, in preferred embodiments the pulses are square waves, but in alternate embodiments may be of any shape, for example, sinusoidal, triangular, or exponential.

Lastly, the waveform is monotonic. That is, the amplitude of successive pulses increases or decreases over time. If the pulse train has n pulses, having amplitudes A₁-Aₙ, then for increasing monotonic pulses:

$${A}_{1} < {A}_{2} < {A}_{3} < {A}_{4} … < {A}_{n}$$

and for decreasing monotonic pulses:

$${A}_{n} < {A}_{n - 1} < {A}_{n - 2} < {A}_{n - 3} … < {A}_{1}$$

The change in amplitude (i.e., the train envelope) between successive pulses can be of any magnitude, for example, in the range of a 10% to 500% increase (for monotonically increasing waveforms) or decrease (for monotonically decreasing waveforms). However, changes that are too small will be effectively perceived as a uniform pulse train and will have little or none of the desired effect. In various embodiments, the train envelope may have, for example, a linear or exponential shape. The upper bound of the amplitude is dependent on the particular application or the particular area of the brain to be stimulated, however, as would be realized, the total energy delivered by each pulse is effectively given by the area under the curve. Therefore, wider pulses may have a smaller upper limit on amplitude and the percent change between pulses, while thinner pulses may have a larger upper limit on amplitude and the percent change between pulses.

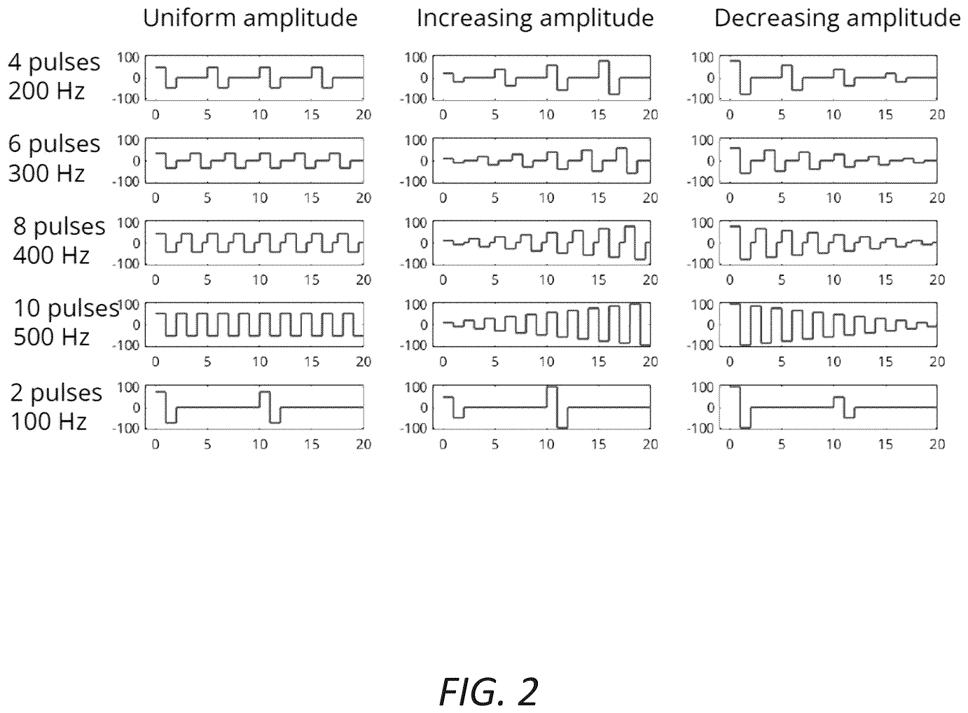

FIG. 2 shows various examples of uniform amplitude waveforms with their corresponding increasing monotonic and decreasing monotonic versions. In one embodiment, the MAPT waveforms may be delivered in bursts of up to 10 pulses over a 5 ms to 100 ms period, depending on the frequency of the pulses.

Various transcranial electrical stimulation techniques, such as high density electrical current stimulation (HD-ECS) can be used to deliver the MAPT waveform and to provide the focality (as small as 1 mm³) and speed (stimulation timing controlled to within <1 μs) needed to elicit the desired neural response. The waveform can be focused and/or steered in real time without physical movement of the electrodes. In one embodiment, the waveform can be focused using a multi-electrode system that injects currents into the brain with a specific set of parameters (i.e., stimulation waveform, current amplitude, and a choice and placement of a subset of electrodes to use on scalp). Specific parameters can be determined, in one embodiment, using a machine learning method such as is described in U.S. patent application Ser. No. 18/021,257, entitled “Method for Focused Transcranial Electrical Current Stimulation”, which is incorporated herein by reference, and which describes a machine learning method for determining the parameters required to focus the stimulation signal.

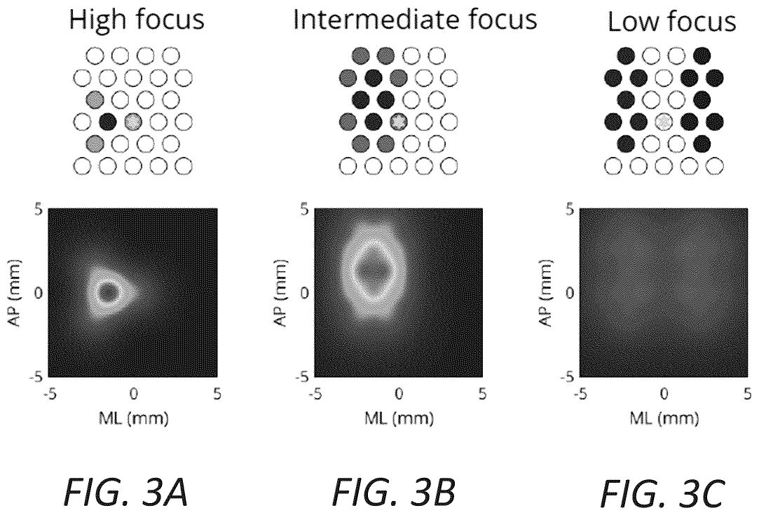

A high density electrode placement on the scalp at the 10-05 standard locations with 256 electrodes can be utilized to achieve the required focality. Aside from the electrode locations, other parameters such as waveforms, amplitude at each electrode, and choice of electrode subset can also be found using the machine learning and optimization techniques mentioned above. The desired focal point, stimulation intensity (current density at the site of stimulation) and electrode placement are provided as inputs to the machine learning algorithm and the other stimulation parameters needed for achieving the desired focality of the stimulation are returned. Various level of focus may be achieved, as shown in FIGS. 3(A-C), which show high, intermediate and low focality respectively in which the upper portion of each figure show transcranial electrode montages and the lower portions of the figures show electric field stimulations in the brain.

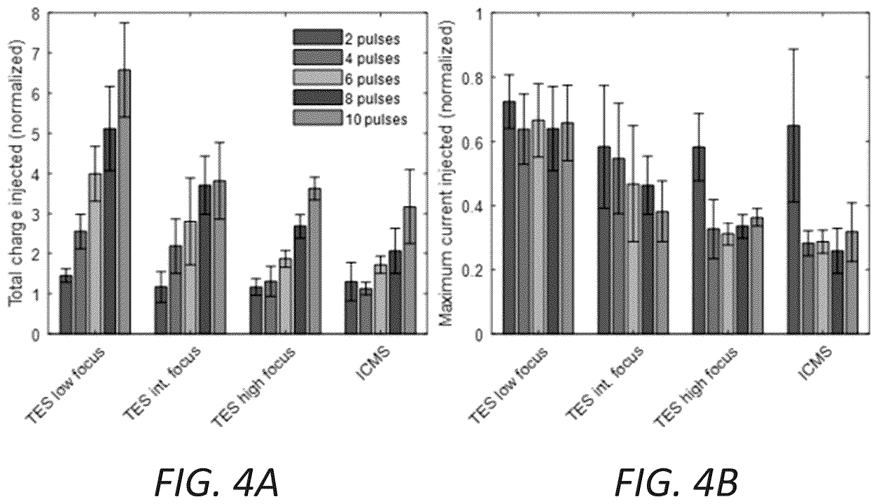

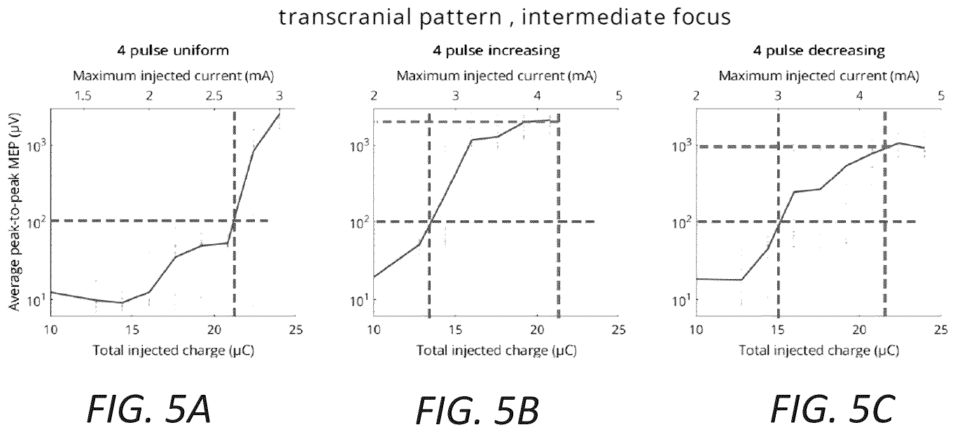

An experiment was conducted on mice to demonstrate the effectiveness of MAPT waveforms. The waveforms were focused on the motor cortex and the motor evoked potentials were measured by EMG. FIGS. 4(A-B) show the effect of the number of pulses delivered as a function of focus. FIG. 4A shows the total charge injected for different numbers of pulses delivered as a function of the focus levels. FIG. 4B shows the maximum current injected as a function of the focus levels. FIGS. 5(A-C) shows the point at which the motor threshold was achieved for a 4-pulse uniform signal, monotonically increasing signal and monotonically decreasing signal, respectively. As can be seen, the motor threshold was achieved at lower total injected charge for both the monotonically increasing signal and monotonically decreasing signal as opposed to the uniform signal, demonstrating the effectiveness of the method.

As would be realized by one of skill in the art, the methods described herein can be implemented on a system comprising a processor and memory, storing software that, when executed by the processor, implements the described methods. The monotonic signal may be generated by a hardware or software pulse generator.

As would further be realized by one of skill in the art, many variations on implementations discussed herein which fall within the scope of the invention are possible. Many combinations of parameters describing the waveforms are possible which are all contemplated to be within the scope of the invention. Moreover, it is to be understood that the features of the various embodiments described herein were not mutually exclusive and can exist in various combinations and permutations, even if such combinations or permutations were not made express herein, without departing from the spirit and scope of the invention. Accordingly, the method disclosed herein is not to be taken as a limitation on the invention but as an illustration thereof. As would be realized, the point of novelty is the use of a monotonic waveform for the purpose of neurostimulation. The scope of the invention is defined by the claims which follow.

1. A method of neurostimulation comprising:
generating a monotonic waveform comprising a train of pulses in which each successive pulse either increases in amplitude from an immediately preceding pulse or decreases in amplitude from an immediately preceding pulse; and
non-invasively delivering the monotonic waveform to the brain of a subject.

2. The method of claim 1 wherein the monotonic waveform is monophasic.

3. The method of claim 2 wherein the monophasic waveform is anodic.

4. The method of claim 2 wherein the monophasic waveform is cathodic.

5. The method of claim 1 wherein the monotonic waveform is biphasic.

6. The method of claim 5 wherein the biphasic waveform is asymmetric.

7. The method of claim 1 wherein each pulse has a duration in the range of 10 us to 10 ms.

8. The method of claim 7 wherein all pulses in the train have identical widths.

9. The method of claim 7 wherein some of the pulses in the train have different widths than other pulses in the train.

10. The method of claim 1 wherein the monotonic waveform has a frequency in the range of 1 Hz to 1 kHz.

11. The method of claim 1 wherein the pulses comprising the monotonic waveform are unevenly spaced in time.

12. The method of claim 1 wherein the pulses comprising the monotonic waveform are rectangular in shape.

13. The method of claim 1 wherein the monotonic waveform has an envelope and further wherein the envelope is linear or exponential in shape.

14. The method of claim 1 wherein each successive pulse increases or decreases in amplitude in the range of 10% to 500% over the previous pulse.

15. The method of claim 1 wherein the monotonic waveform is delivered in bursts of between 2 and 10 pulses.

16. The method of claim 15 wherein the bursts of between 2 and 10 pulses are delivered over a time period between 5 ms and 100 ms.

17. The method of claim 1 wherein the monotonic waveform is focused to a specific area of the brain.

18. The method of claim 1 wherein the monotonic waveform is delivered via a transcranial electrical stimulation technique.

19. The method of claim 1 wherein the monotonic waveform is delivered via a plurality of electrodes placed on the skull of the subject.

20. A system comprising:
a processor;
a pulse generator; and
software that, when executed by the processor, controls the pulse generator to implement the method of claim 1.
